(19) United States
(12) Patent Application Publication
(10) Pub. No.: US 2014/0017711 A1
(43) Pub. Date: Jan. 16, 2014

(54) METHODS OF DIAGNOSING ULCERATIVE COLITIS AND CROHN'S DISEASE

(71) Applicants: Kent D. Taylor, Ventura, CA (US); Dermot P. McGovern, Los Angeles, CA (US); Jerome I. Rotter, Los Angeles, CA (US); Stephan R. Targan, Santa Monica, CA (US); Talin Haritunians, Los Angeles, CA (US)

(72) Inventors: Kent D. Taylor, Ventura, CA (US); Dermot P. McGovern, Los Angeles, CA (US); Jerome I. Rotter, Los Angeles, CA (US); Stephan R. Targan, Santa Monica, CA (US); Talin Haritunians, Los Angeles, CA (US)

(73) Assignee: CEDARS-SINAI MEDICAL CENTER, Los Angeles, CA (US)

(21) Appl. No.: 14/007,381

(22) PCT Filed: Mar. 26, 2012
(86) PCT No.: PCT/US12/30611

§ 371 Date: Sep. 25, 2013

Related U.S. Application Data
(60) Provisional application No. 61/467,872, filed on Mar. 25, 2011.
(60) Provisional application No. 61/467,893, filed on Mar. 25, 2011.

(51) Int. Cl. G01N 33/68

(52) U.S. Cl. CPC G01N 33/6893
USPC 435/7.92

(57) ABSTRACT

The present invention relates to methods of prognosing inflammatory bowel disease (IBD) in an individual by determining the presence of at least one risk genetic variant and/or at least one risk serological marker. In one embodiment, the presence of risk serological marker ANCA is indicative of an aggressive form of ulcerative colitis. In another embodiment, the present invention relates to methods of diagnosing a Crohn's disease subtype in an individual, where the presence of risk variants and serological markers I2, OmpC and/or Cbir1 are indicative of the Crohn's disease subtype.

### FIELD OF INVENTION

The invention relates to the field of genetics and medicine. More specifically, the invention relates to methods of diagnosing and prognosing inflammatory bowel disease including ulcerative colitis and Crohn's disease.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)), Genetic factors play an important role in IBD pathogenesis, as evidenced by the increased rates of MD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet 29, 223 (2001); V. D, Peltekova et al., Nat Genet 36, 471 (2004)). CD and UC are thought to be related disorders that share some genetic susceptibility loci but differ at others.

### SUMMARY OF THE INVENTION

Various methods include a method of prognosing inflammatory bowel disease (IBD) in an individual, comprising obtaining a sample, assaying the sample to determine the presence or absence of one or more risk variants at Chromosome 4, assaying the sample to determine the presence or absence of serological marker ANCA, prognosing an aggressive form of inflammatory bowel disease in the individual based on the presence of one or more risk variants at Chromosome 4 and the presence of serological marker ANCA. In another embodiment, the aggressive form of inflammatory bowel disease is characterized by an aggressive form of ulcerative colitis. In another embodiment, the one or more risk variants at Chromosome 4 are at the genetic loci of AFP, AFM, RASSF6 and/or PGM2. In another embodiment, the one or more risk variants at Chromosome 4 comprise SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, and/or SEQ. ID. NO.: 16. In another embodiment, the presence of serological marker ANCA comprises a high level of serological marker ANCA as compared to a healthy subject. In another embodiment, the absence of serological marker ANCA is indicative of inflammatory bowel disease with Crohn's like conditions.

Other embodiments include a method of diagnosing an ulcerative colitis subtype in an individual, comprising obtaining a sample, assaying the sample to determine the presence or absence of serological marker ANCA, and diagnosing the ulcerative colitis subtype in the individual, wherein the presence of serological marker ANCA is indicative of an aggressive subtype of ulcerative colitis, and wherein the absence of serological marker ANCA is indicative of an ulcerative colitis subtype with Crohn's disease characteristics. In another embodiment, further further comprising the presence of one or more risk variants at Chromosome 4. In another embodiment, further comprising SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, and/or SEQ. ID. NO.: 16. in another embodiment, the presence of serological marker ANCA comprises a high level of serological marker ANCA as compared to a healthy subject.

Other embodiments include a method of diagnosing a Crohn's disease subtype in an individual; comprising obtaining a sample, assaying the sample to determine the presence or absence of one or more genetic risk variants located at Chromosome 15, Chromosome 18, and/or AK097193 genetic locus, and assaying the sample to determine the presence or absence of serological markers I2, OmpC and/or Cbir1, diagnosing the Crohn's disease subtype based on the presence of one or more genetic risk variants and the presence of one or more serological markers. In another embodiment, the one or more genetic risk variants comprise SEQ. ID. NO.: 17, SEQ. ID. NO.: 18, SEQ. ID. NO.: 19, and/or SEQ. ID. NO.: 20. In another embodiment, SEQ. ID. NO.: 17 is associated with the presence of antibody I2. In another embodiment, SEQ. ID. NO.: 18 is associated with the presence of antibody OmpC. In another embodiment, SEQ. ID. NO.: 19 and/or SEQ. ID. NO.: 20 is associated with the presence of antibody Cbir1.

Various embodiments herein also include a method of diagnosing susceptibility to Crohn's disease in an individual, comprising obtaining a sample, assaying the sample to determine the presence or absence of one or more genetic risk variants located at the genetic loci of FHIT, ETV4, ME1, WDR64, A2BP1, CDH2, HSPBP1, PPP6R1, and/or BRSK1, diagnosing susceptibility to Crohn's disease in the individual based on the presence of one or more genetic risk variants. In another embodiment, the one or more genetic risk variants are associated with the presence of serological marker ANCA. In another embodiment, the one or more genetic risk variants include SEQ. ID. NO. 21, SEQ. ID. NO.: 22, SEQ. ID. NO.: 23, SEQ. ID. NO.: 24, SEQ. ID. NO.: 25, SEQ. ID. NO.: 26, and/or SEQ. ID. NO.: 27.

Other features and advantages of the invention will become apparent from the following detailed description, taken in conjunction with the accompanying drawings, which illustrate, by way of example, various embodiments of the invention.

### BRIEF DESCRIPTION OF THE FIGURES

FIG. 1 depicts two Chr.4 loci associated with the phenotypic difference of UC patients, one for UC severity as typified by ANCA level, the other for the expression of antibodies more characteristic of CD. If a listed SNP allele has an OR>1, it means that this allele's presence is associated with the UC subtype; if OR<1, it means this allele's absence is associated with the UC subtype. As an example, rs7668327 is a G/C SNP and the table shows the G allele with OR<1, so that the absence of G (i.e., the presence of C) is associated with the ANCA UC subtype.

FIG. 2 depicts −log(10)p-values of CD ANCA analysis. FIG. 2(a) depicts definition of ANCA phenotype. FIG. 2(b) depicts QQ plot. The ANCA analysis compared the lowest and highest tertile with the center tertile removed.

FIG. 3 depicts antibody distribution divided into tertiles and scores are then summed FIG. 3(a) depicts ASCA scores. FIG. 3(b) depicts anti-Cbir1 scores. FIG. 3(c) depicts anti-I2 scores. FIG. 3(d) depicts anti-OmpC scores.

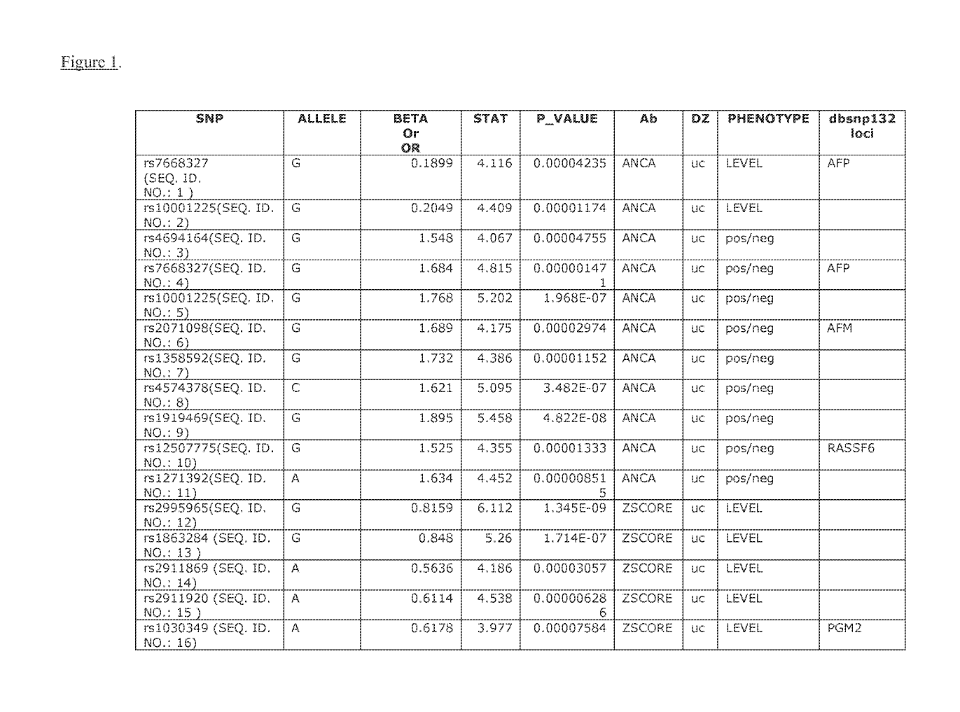

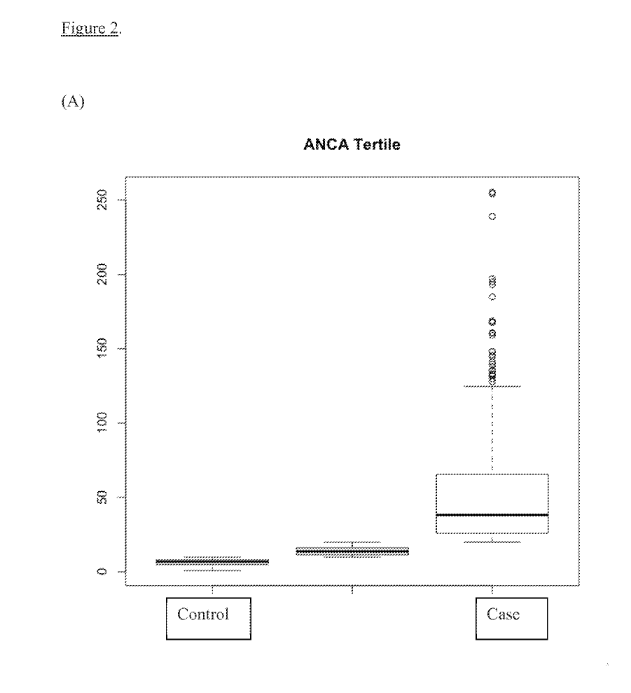

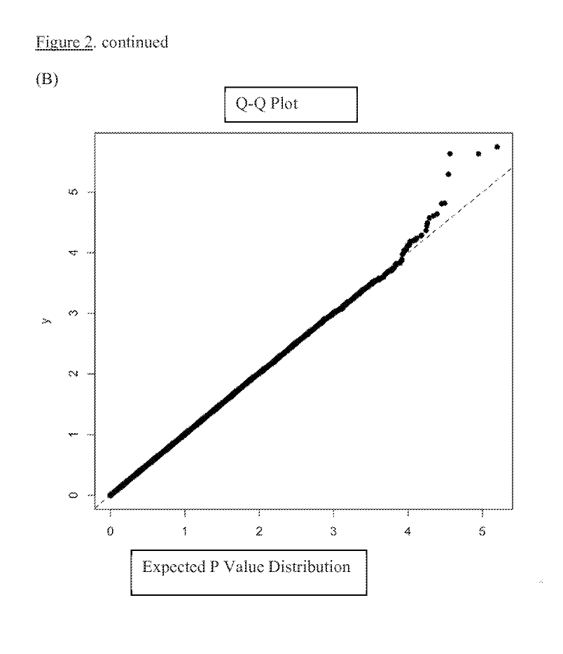

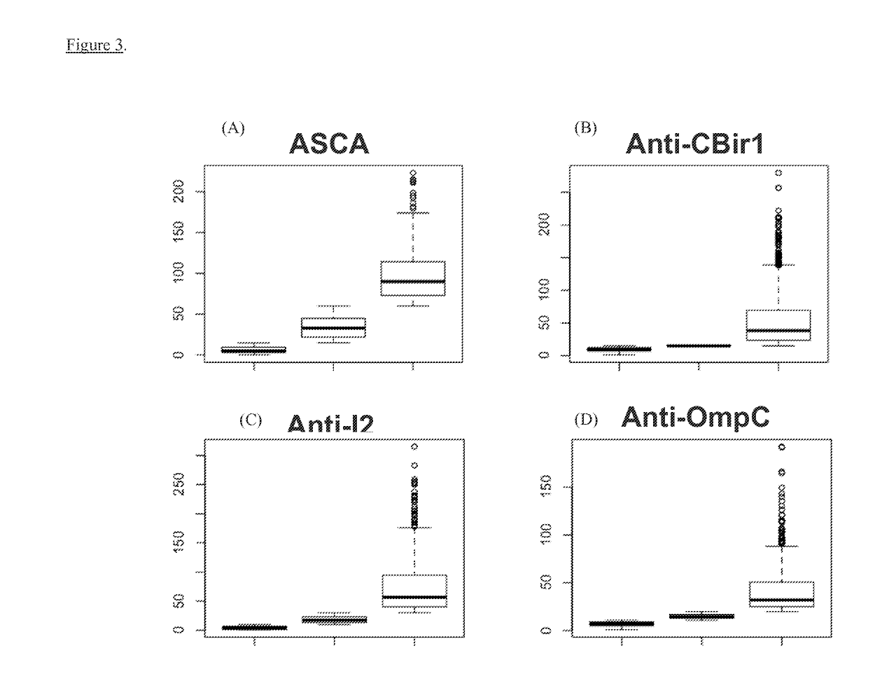

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, N.Y. 2001); March, Advanced Organic Chemistry Reactions, Mechanisms and Structure 5th ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“IBD” as used herein is an abbreviation of inflammatory bowel disease.

“CD” as used herein is an abbreviation of Crohn's Disease.

“SNP” as used herein is an abbreviation of single nucleotide polymorphism.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

As disclosed herein, it has been found that response to ANCA has been associated with more aggressive disease behavior in ulcerative colitis (UC) patients, whereas sero-reactivity to ASCA, anti-CBir1, anti-I2, and anti-OmpC have been particularly associated with subtypes of Crohn's disease. There is also a hereditary component to expression of these antibodies. The inventors assessed the genetic contribution to TBD associated serological profiles UC cases, with 1327 UC cases genotyped with the Illumina CNV370 or OmniExpress beadchips, and were sero-typed for ANCA, ASCA, anti-CBir1, anti-I2, and anti-OmpC by ELISA. The inventors performed regression analyses, adjusted for population stratification using principal components as covariates, testing for an association of UC with antibody response. A Z-score for ASCA, anti-CBir1, anti-I2, and anti-OmpC together was generated by adding the four Z-scores for each individual antibody for each subject. The Z-scores were calculated from within the UC cohort only. Association of UC was assessed with this combined score and with ANCA status alone.

As further disclosed herein, the results demonstrate two genome-wide significant associations with UC and (1) ANCA at chr.4 (rs1919469 p_{logistic}=4.82×10⁻⁸, OR=1.90; rs10001225 p_{logistic}=1.97×10⁻⁷, OR=1.77). An additional three SNPs within this region are also found to be associated with nominal significance (p<10⁻⁵); and (2) at a second region on chr.4˜37 Mb away, with the combined ASCA, I2, CBir1 and Ompc Z-score (rs2995965 pₗᵢₙₑₐᵣ=1.35×10⁻⁹, β=0.82; rs1863284 pₗᵢₙₑₐᵣ=1.71×10⁻⁷, β=0.85; rs2911920 pₗᵢₙₑₐᵣ=6.29×10⁻⁶, β=0.60. RELL1, a homologue of RELT the TNF receptor that induces epithelial cell apoptosis is located at this locus. In general, these and additional observations disclosed herein support two chromosome 4 loci associated with the phenotypic differences of UC patients, one for UC severity as typified by ANCA level, the other for the expression of antibodies more characteristic of CD.

In one embodiment, the present invention provides a method of diagnosing susceptibility to inflammatory bowel disease (IBD) in an individual by obtaining a sample from the individual, assaying the sample to determine the presence or absence of one or more risk variants on Chromosome 4, and/or risk serological markers, where the presence of one or more risk variants at Chromosome 4 and/or risk serological markers is indicative of susceptibility to IBD in the individual. In another embodiment, the IBD is ulcerative colitis. In another embodiment, the one or more risk variants on Chromosome 4 include SEQ. ID. NOS.: 1-16. In another embodiment, the risk serological markers include ANCA, ASCA, I2, Cbir1, and/or OmpC.

In one embodiment, the present invention provides a method of diagnosing an inflammatory bowel disease (IBD) subtype in an individual, by obtaining a sample from the individual, assaying the sample to determine the presence of one or more risk variants on Chromosome 4 and one or more risk serological markers, and diagnosing the IBD subtype in the individual based on the presence of one or more risk variants on Chromosome 4 and one or more risk serological markers. In another embodiment, the presence of SNP rs1919469 and/or rs10001225 on Chromosome 4 and ANCA is indicative of ulcerative colitis. In another embodiment, the ulcerative colitis is an aggressive form of the disease. In another embodiment, the presence of SNPs rs2995965, rs1863284, and/or rs2911920 on Chromosome 4 and risk serological markers ASCA, I2, Cbir1, and/or OmpC is indicative of Crohn's disease.

In one embodiment, the present invention provides a method of prognosing inflammatory bowel disease (IBD) in an individual, by obtaining a sample from the individual, assaying the sample to determine the presence of one or more risk variants and/or risk serological markers, and prognosing a severe form of IBD based on the presence of one or more risk variants and/or risk serological markers. In another embodiment, the present invention provides a method of prognosing a severe form of ulcerative colitis, by determining the presence or absence of one or more risk variants at Chromosome 4 and ANCA expression, where the presence of one or more risk variants at Chromosome 4 and a high level of ANCA expression relative to a normal subject are indicative of the severe form of ulcerative colitis.

In one embodiment, the present invention provides a method of treating IBD in an individual by determining the presence of one or more risk variants at Chromosome 4 and one or more risk variants, and treating the individual.

As disclosed herein, the inventors conducted a genome-wide association study (GWAS) on 1544 CD subjects serotyped for CD-associated antibodies (ASCA, anti-CBir1, anti-I2, and anti-OMPC). Serum antibody expression was measured by ELISA and levels were log transformed prior to analyses. Single nucleotide polymorphism (SNP) data were generated using Illumina technology (˜550K SNPs with MAF>0.05) at Cedars-Sinai Medical Center. Adjustment for population stratification was carried out using two principal components as covariates in the analyses (Eigensoft). The significance of association was tested using logistic regression for antibody positive or negative and linear regression for antibody level after transformation. To overcome multiple testing issues significance was defined to be p<2e-07.

As further disclosed herein, at the pre-defined level of significance, the inventors observed two significant associations: 1) expression of anti-I2 was significantly associated with three SNPs spanning 90 kb of chr. 15 that included the 3 region of human EST BF729345, among other ESTs (rs246336, OR for G allele and anti-I2 positivity, 1.8; p (logistic regression)=8.6e-08); and 2) Expression of anti-OMPC was significantly associated with rs6566234 on chr. 18 (beta coefficient for C allele was −0.28, p (linear regression)=1.4e-07), potentially in LD with CDH19. In addition, 3) anti-Cbir1 positivity was associated with gene AK097193 on chr. 1 (rs1022265 G allele OR for anti-CBir positivity 0.68 p (logistic regression)=7.6 e-07); and 4) ASCA positivity was associated with two SNPs on chr. 3 (rs291528 & rs291523, OR 1.9, p (logistic regression)=5e-07).

In one embodiment, the present invention provides a method of diagnosing susceptibility to a Crohn's disease subtype in an individual, by obtaining a sample from the individual, assaying the sample to determine the presence or absence of risk genetic variants and/or one or more risk serological markers, where the presence of one or more risk genetic variants and/or one or more risk serological markers are indicative of susceptibility to the Crohn's disease subtype. In another embodiment, the Crohn's disease subtype is associated with an aggressive form of the disease. In another embodiment, the one or more risk genetic variants are located on Chromosome 15 and the one or more risk serological markers include I2. In another embodiment, the one or more risk genetic variants are located on Chromosome 18 and the one or more risk serological markers include OmpC. In another embodiment, the one or more genetic variants are located on the AK097193 genetic locus and the one or more risk serological markers include Cbir. In another embodiment, the one or more genetic variants are located on Chromosome 3 and the one or more risk serological markers include ASCA.

In one embodiment, the present invention provides a method of prognosing an aggressive form of Crohn's disease in an individual by obtaining a sample from the individual, assaying the sample to determine the presence of one or more risk genetic variants and/or risk serological markers, and prognosing a severe form of Crohn's disease based on the presence of one or more risk genetic variants and/or risk serological markers.

In one embodiment, the present invention provides a method of treating Crohn's disease in an individual by obtaining a sample from the individual, assaying the sample to determine the presence of one or more risk genetic variants and/or risk serological markers, and treating the individual.

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase. Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Tag polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature”, Nucleic Acids Research 28:655-661 (2000)). Minor grove binders include, but are not limited to, compounds such as dihydrocyclopyrroloindole tripeptide (DPI,).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al.,(Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well-known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

Ulcerative colitis (UC), a subtype of Inflammatory Bowel Disease (IBD), is a chronic inflammatory condition of the gastrointestinal tract with a complex genetic and environmental component. UC particularly, the environmental factors and the role of bacteria in diseases pathogenesis remains unknown. Response to ANCA has been associated with more aggressive disease behavior in UC patients, whereas sero-reactivity to ASCA, anti-CBir1, anti-I2, and anti-OmpC have been particularly associated with subtypes of Crohn's disease. Furthermore, there is a hereditary component to expression of these antibodies.

The inventors assessed the genetic contribution to IBD associated seroloical profiles in UC cases. 1327 UC cases were genotyped with the Illumina CNV370 or OmniExpress beadchips, and were sero-typed for ANCA, ASCA, anti-CBir1, anti-I2, and anti-OmpC by ELISA. Regression analyses was performed, adjusted for population stratification using principal components as covariates, testing for an association of UC with antibody response. A Z-score for ASCA, anti-CBir1, anti-I2, and anti-OmpC together was generated by adding the four Z-scores for each individual antibody for each subject. The Z-scores were calculated from within the UC cohort only. Association of UC was assessed with this combined score and with ANCA status alone.

The results demonstrate two genome-wide significant associations with UC and (1) ANCA at chr.4 (rs1919469 p_{logistic}=4.82×10⁻⁸, OR=1.90; rs10001225 p_{logistic}=1.97×10⁻⁷, OR=1.77). An additional three SNPs within this region are also found to be associated with nominal significance (p<10⁻⁵); and (2) at a second region on chr.4˜37 Mb away, with the combined ASCA, I2, CBir1 and Ompc Z-score (rs2995965 pₗᵢₙₑₐᵣ=1.35×10⁻⁹, β=0.82; rs1863284 p^{linear}=1.71×10⁻⁷, β=0.85; rs2911920 pₗᵢₙₑₐᵣ=6.29×10⁻⁶, β=0.61), RELL1, a homologue of RELT the TNT receptor that induces epithelial cell apoptosis is located at this locus. These observations support that these two loci contribute to the phenotypic difference of UC patients, one for UC severity as typified by ANCA level, the other for the expression of antibodies more characteristic of CD.

### Example 2

It has been reported that CD patients can be characterized by the association of disease phenotypes with the expression of antibodies to microbial antigens. For example CD associated serologies such as ASCA, I2, CBir1 and OMPc are associated with a more aggressive course of disease and an increased chance of surgery. It has also been previously demonstrated the heritable nature of these IBD associated antibodies.

The inventors conducted a genome-wide association study (GWAS) on 1544 CD subjects serotyped for CD-associated antibodies (ASCA, anti-CBir1, anti-I2, and anti-OMPC). Serum antibody expression was measured by ELISA and levels were log transformed prior to analyses. Single nucleotide polymorphism (SNP) data were generated using Illumina technology (˜550K SNPs with MAF>0.05) at Cedars-Sinai Medical Center. Adjustment for population stratification was carried out using two principal components as covariates in the analyses (Eigensoft). The significance of association was tested using logistic regression for antibody positive or negative and linear regression for antibody level after transformation. To overcome multiple testing issues significance was defined to be p<2e-07.

At the pre-defined level of significance, the inventors observed two significant associations: 1) expression of anti-I2 was significantly associated with 3 SNPs spanning 90 kb of chr. 15 that included the 3 region of human EST BF729345, among other ESTs (rs246336, OR for G allele and anti-I2 positivity, 1.8; p (logistic regression)=8.6e-08); and 2) Expression of anti-OMPC was significantly associated with rs6566234 on chr. 18 (beta coefficient for G allele was −0.28, p (linear regression)=1.4e-07), potentially in LD with CDH19. In addition, 3) anti-CBir1 positivity was associated with gene AK097193 on chr. 1 (rs1022265 G allele OR for anti-CBir positivity 0.68 p (logistic regression)=7.6 e-07); and 4) ASCA positivity was associated with two SNPs on chr. 3 (rs291528 & rs291523, OR 1.9, p (logistic regression)=5e-07).

Examples of rs246336, rs6566234, rs291528, and rs291523 are provided herein as SEQ. ID. NO.: 17, SEQ. ID. NO.: 18, SEQ. ID. NO.: 19, and SEQ. ID. NO.: 20, herein.

These results show that GWAS of serum expression to microbial antibodies may lead to discovery of novel loci affecting CD course and thus targets for therapies for aggressive CD.

### Example 3

Crohn's Disease (CD), a subtype of Inflammatory Bowel Disease (IBD), is a chronic inflammatory condition of the gastrointestinal tract with a complex genetic and environmental component. It has been reported that combinations of genetic and serological markers, including antibodies to anti-Saccharomyces cerevisiae (ASCA), E. Coli outer membrane porinC (OmpC), Pseudomonas fluorescens protein (I2), and anti-flagellin (CBir1), are associated with complications of Crohn's disease. Severe CD is associated with the expression of more than one antibody as well as higher levels of antibody expression.

The inventors identified genes contributing to CD severity by conducting a genome-wide association study (GWAS) of antibody expression in serotyped CD subjects. 1537 CD cases with complete serum antibody profile were genotyped with the Illumina 610quad or OmniExpress beadchips at Cedars-Sinai Medical Center Medical Genetics Institute. Serum antibody for ANCA, ASCA, anti-OmpC, anti-I2, and anti-CBir1 expression was measured by ELISA and log-transformed prior to analyses. 303,147 SNPs with HWE>0.001, MAF>0.02, and GENO>0.02 (genotyping rate=0.9993) were included in analyses. Association for autosomal chromosomes of CD was assessed with Antibody Score using linear regressgion and with ANCA status using logistic regression.

An excess of significance in the tail of the distribution suggests that true positive results are present (below).

TABLE 1
SNPs associated with ANCA positive/negative

| CHR | SNP | Allele | OR | P | GENE(S) IN LD |
|---|---|---|---|---|---|
| 3 | rs1973780 | A | 1.63 | 3.6 × 10⁻⁷ | FHIT |
| | (SEQ. ID. | | | | Fragile histidine triad gene |
| | NO.: 21) | | | | |
| 17 | rs1728171 | A | 0.45 | 3.2 × 10⁻⁶ | ETV4 |
| | (SEQ. ID. | | | | ETS variant 4 |
| | NO.: 22) | | | | |
| 6 | rs9449593 | A | 2.1 | 7.7 × 10⁻⁶ | ME1 |
| | (SEQ. ID. | | | | Malic enzyme 1 |
| | NO: 23) | | | | |
| 1 | rs6690359 | A | 0.65 | 9.3 × 10⁻⁶ | WDR64 |
| | (SEQ. ID. | | | | WD repeat domain 64 |
| | NO.: 24) | | | | |

TABLE 2
SNPs associated with Antibody Score in Crohn's Disease

| CHR | SNP | Allele | OR | P | GENE(S) IN LD |
|---|---|---|---|---|---|
| 16 | rs1019257 | A | −0.60 | 2.2 × | A2BP1 (aka RBFOX1) |
| | (SEQ. ID. | | | 10⁻⁶ | Ataxin 2 binding protein 1 |
| | NO.: 25) | | | | Trans-golgi network; |
| | | | | | associated with osteoarthritis |
| | | | | | and aortic plaque |
| 18 | rs766613 | A | 0.37 | 3.6 × | CDH2 |
| | (SEQ. ID. | | | 10⁻⁶ | Cadherin 2 |
| | NO.: 26) | | | | |
| 19 | rs10403164 | A | −0.37 | 9.8 × | HSPBP1, PPP6R1, BRSK1 |
| | (SEQ. ID. | | | 10⁻⁶ | Heat shock 70 kDa binding |
| | NO.: 27) | | | | protein, co-chaperone 1; |
| | | | | | Protein phophatase |
| | | | | | 6 regulatory subunit 1; BR |
| | | | | | serine/threonine kinase |

These results show genes for antibody expression in CD subjects. These genes are novel with respect to current GWAS results for CD. Because antibody expression is further associated with disease severity, characterization of these genetic associations may add to the list of genetic determinants of CD as well as to the characterization of immune processes that affect CD phenotype.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method of prognosing inflammatory bowel disease (IBD) in an individual, comprising:
obtaining a sample from the individual;
assaying the sample to determine the presence or absence of one or more risk variants at Chromosome 4;
assaying the sample to determine the presence or absence of serological marker ANCA; and
prognosing an aggressive form of inflammatory bowel disease in the individual based on the presence of one or more risk variants at Chromosome 4 and the presence of serological marker ANCA.

2. The method of claim 1, wherein the aggressive form of inflammatory bowel disease is characterized by an aggressive form of ulcerative colitis.

3. The method of claim 1, wherein the one or more risk variants at Chromosome 4 are at the genetic loci of AFP, AFM, RASSF6 and/or PGM2.

4. The method of claim 1, wherein the one or more risk variants at Chromosome 4 comprise SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, and/or SEQ. ID. NO.: 16.

5. The method of claim 1, wherein the presence of serological marker ANCA comprises a high level of serological marker ANCA as compared to a healthy subject.

6. The method of claim 1, wherein the absence of serological marker ANCA is indicative of inflammatory bowel disease with Crohn's like conditions.

7. A method of diagnosing an art ulcerative colitis subtype in an individual, comprising:
obtaining a sample from the individual;
assaying the sample to determine the presence or absence of serological marker ANCA; and
diagnosing the ulcerative colitis subtype in the individual,
wherein the presence of serological marker ANCA is indicative of an aggressive subtype of ulcerative colitis, and
wherein the absence of serological marker ANCA is indicative of an ulcerative colitis subtype with Crohn's disease characteristics.

8. The method of claim 7, further comprising assaying the sample to determine the presence of one or more risk variants at Chromosome 4.

9. The method of claim 8, wherein the one or more risk variants at Chromosome 4 comprise SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 10, SEQ. ID. NO.: 11 SEQ. ID. NO.: 12, SEQ. ID. NO.: 13, SEQ. ID. NO.: 14, SEQ. ID. NO.: 15, and/or SEQ. ID. NO.: 16.

10. The method of claim 7, wherein the presence of serological marker ANCA comprises a high level of serological marker ANCA as compared to a healthy subject.

11. A method of diagnosing a Crohn's disease subtype in an individual; comprising:
obtaining a sample from the individual;
assaying the sample to determine the presence or absence of one or more genetic risk variants located at Chromosome 15, Chromosome 18, and/or AK097193 genetic locus; and
assaying the sample to determine the presence or absence of serological markers I2, OmpC and/or Cbir1; and
diagnosing the Crohn's disease subtype based on the presence of one or more genetic risk variants and the presence of one or more serological markers.

12. The method of claim 11, wherein the one or more genetic risk variants comprise SEQ. ID. NO.: 17, SEQ. ID. NO.: 18, SEQ. ID. NO.: 19, and or SEQ. ID. NO.: 20.

13. The method of claim 12, wherein SEQ. ID. NO.: 17 is associated with the presence of antibody I2.

14. The method of claim 12, wherein SEQ. ID. NO.: 18 is associated with the presence of antibody OmpC.

15. The method of claim 12, wherein SEQ. ID. NO.: 19 and/or SEQ. ID. NO.: 20 is associated with the presence of antibody Cbir1.

16. A method of diagnosing susceptibility to Crohn's disease in an individual, comprising:
obtaining a sample from the individual;
assaying the sample to determine the presence or absence of one or more genetic risk variants located at the genetic loci of FHIT, ETV4, ME1, WDR64, A2BP1, CDH2, HSPBP1, PPP6R1, and/or BRSK1; and
diagnosing susceptibility to Crohn's disease in the individual based on the presence of one or more genetic risk variants.

17. The method of claim 16, wherein the one or more genetic risk variants are associated with the presence of serological marker ANCA.

18. The method of claim 16, wherein the one or more genetic risk variants comprise SEQ. ID. NO. 21, SEQ. ID. NO.: 22, SEQ. ID. NO.: 23, SEQ. ID. NO.: 24, SEQ. ID. NO.: 25, SEQ. ID. NO.: 26, and/or SEQ. ID. NO.: 27.


SEQUENCE LISTING (27 sequences)

SEQ ID NO: 1 - DNA, 601 bp, Homo sapiens
agaaaaggcaaaagtggggagaaagcactaaaacgggagacaagttaaaatttctttttaattgataggtcacgttctcactctatttgcctttaagggaagaaagcaatcaagttaatatgttttccttcattgtatagtatgtaactacggacactattagaggagggatttgtgtagcacttaggacattatacttgataatttccaagggtctttctagatttaaaagtctgattctaacgtagtaataaaaataaaggcccaattttctctttaatattgcctgaagatattactstattattgcattaaaattaaacattcacacattgtttgcactgctaaataaaattatgtaatttcttcttctttccttcctccttcccccatccctctctatttccctttccccttccttctttcctggccttttttccttctttctttgttcccttctccctccctcccctttcttcctttttctaaagctggctttgagatcctttattaaagaataaatctttaaaacttatactttattttccctgttgcaggaaaaaatcatgtcctacatatgttctcaacaagacactctgtc

SEQ ID NO: 2 - DNA, 874 bp, Homo sapiens
cagactcagtgcattaaatgccatctagatgttgacaacacatgtttatagctccagcctggatttcttccttgacttacaggctcttacgtcagcttcctccttaacatctccatgctgataatgtatagacaacacaaacttagcacattacaaattgaatgtgaatctactccttgtcccaaatttgtacctccatcttgtctatctgtctgtttttatgagtcagacattttggatttattgatgacccctctcttgcatatcacccacacattcaatctatgagcaaagcttgtcggctctacctttaaaaagtatccagaatttgactatttctcaatactttggtaccagtcacaatcatatgcctcactggggttattgcaatagattcctaattggtctttttacaagtgaccttgctccaccctcaaccctattctcaaaagtgaacattatgtcacttttccactcagaatcctctagtggcttcccacytctctcagagtaaaatgcaaagtctgtccaatgaccctacatgataatatctttctgaccttttttgttactcctctgatcaggtacagaggtctccctgctgctcttcaagttaccaatcaatgtcctgctcagggttttgccctcatgacccttctggcttagaagaccctcagctatgtgtgaggctcacttcatcaccaacttcaagcttctactcgtagttatctgctaaatgatgatgttttcctcaactaccttattttaattcagaaatgccattcccagtccccacaacacttatcaccatctgacaaacactagattttacttgtttattttctgtctacctcactagagtgtaagctccatg

SEQ ID NO: 3 - DNA, 1001 bp, Homo sapiens
ctattatgttctctgcctcaagtaccagctctgacaacacatgggcagactgggaactaatgtccccagaagcaatctcttcaactagtgagagatggagctggtgatagttcttaagtgtctccagtggtgtgaagtctcaggtgcccacagtagtgacccacctatcagcacactcttaattggctttcttctcttccttgcctcatcttcttatttccttactctgctttcagggattgccttccaaatagactctttctacccaattctggttttagagttggttttggaagaaaccaaaccaagacaatggtcaagcaagtttatttctccatgagagccttttgaatagaaacctgaatcttgtgagggagtaagcctcaggaatatttgaaggaggagaaactaacaaatccaaagggcttgaggtgggaacatacttggcaattttaaagaatagcaagaaggccatgtggctggagtggggcatgtgaaggyagagagttaggagatgccttcagagagttaggctggggctgatccttgtgggcgatgatagagatttaggattttattctcagtgtcatgtgaaaacattggatggttgaggtgggcattaatgtaatatgatttaagttttaaaatattctggaaattaagtgtagcatattgagcatacatatttatcttttgtgcaataaacatataaagacaaagagaactagaaaggaagtcacaacgcggaagaagtattcaacaacattttgaaatctagaaatatagaaaagacctaacagatttaacagactagagaaagttgaaacctaagagaagtgggggagagagaggctgttataggttgaattgtgtcccctaaaagatgatgtgttgtggtcctaacctctgctacctcagaacatgaccttatttagaacaagagtctttgcagatttagtcatgttaacatgaagtcattaggtgggccttaatccaatata

SEQ ID NO: 4 - DNA, 601 bp, Homo sapiens; identical to SEQ ID NO: 1

SEQ ID NO: 5 - DNA, 874 bp, Homo sapiens; identical to SEQ ID NO: 2

SEQ ID NO: 6 - DNA, 801 bp, Homo sapiens
ttcttccttttaccttgttttacaacttgctcttcttgtctctttctattttttcttttccttcccatcttaccctcccttcccttcccttcccttcccttgtctacattcattgcttcctgtttcttccctcacccgtcttctctctttcatttttattttttatagctttttatatgaagttgccagaaggaacccatttgtcttcgcccctacacttctaactgttgctgttcattttgaggaggtggccaaatcatgttgtgaagaacaaaacaaagtcaactgccttcaaacaagggtgggtatagcatttgttccatgaagaggataagaaatcactcaataccacagatccagaccctgacttatattatagcaaaaggcttgcttaccatatrtgatgttctcttgtcacattcagtgattttcaaaccattggtcaccaggtgctgatctaaatattctctttacttgagactcttgaatgacagccagtcattttcagggtctctcttccagctttgttttctttgtaaattggaaggaagccctggtgagtgaatgcccagggtgaatgatgacattttatcttttatctttgacaatttgtagtcttttgactacaaacaacatggtataatctcagtagggatggccaagctcacagcatgctgccccaccaagggagcacttatgcacctttttaccaagtgcaattcaatcatcagcaagactggtcctacctttgtgatgtgccgtaagtgcatatttcttttaatatgtgtgtttactgaaata

SEQ ID NO: 7 - DNA, 605 bp, Homo sapiens
tttgccaagagtgtttatggattacttaattataaagtttgcatgttgaaatcttaaagaaacaagcagattgacatgacttgtttaaaaatgcagtattcattaatgaattcaatccatcagcaatatttcttcattgtcatagatttcctcagttttatcagggccatagttgcttggaaaaaatattcaccagaaaaaggtttctttttaatagtaattacttggaaagaatataaagttcttcatttctctcaaatgatagaacactttccataaaggttctcacgaagtaatgaagactgctaaacccacatcttttcttgttttcttcagctrcattaaatgtcaatctcgttagagaagtttactagatgcgggtggaaaatcctatttacaatttacatattccaggtataaggataagataagtaagccacaatgactactacagtgtacatgagaaggcttatctttttcagaggtcattactgtaaatagaatcattgaaaattgcaagacattaatttgtggaatgaatatacagagagagctgatatattgaagcaacacacacaggaatattccactttagcctcacttcc

SEQ ID NO: 8 - DNA, 501 bp, Homo sapiens
tgattacaacagcctgagtcactaagtacattttatagatggtagagtattgagtttcaagaggttcaagtgacttgcctgatgtcacacagtcagtaaggacagggctaagtgtgaaattcaggccaactgtttgcaaaaacaataacaattttccctataacagccactgctcgttaggataaggtggactaggtctgactaattaagctgataaccagcgctcttgctaaacaactaagcctacaagkttattggtaccttcccactggaatcttatattcatggttcaagaactctgaatttgtctttctctcgagaatccatttaattcacttcaatttatattcatggagtattttactaaatactaatattttactaaataaaaagtatatgcttttattaaatcatggcttcttaatatgtatctattactattactattactaaatactaaatattttactaaataaaaagtatatgcttttattaaatcat

SEQ ID NO: 9 - DNA, 601 bp, Homo sapiens
gggttccttttccagtttcaagtcagatccacagagcaaatttcgcattttcatcaccaagaagagagctaaacagatatattagttatttgcctgtgggaacatacaccctttcttgtgggcagggagcctgggaaatcccctcgtatttcaaaaattcaattgtcaaaaaacaaaagccaattttgagtaatctttgagattttactataattctgtgtcaaagcagagtattgaggaaaagtttagtttgctatattagcctaagatattgagcttaaattaaatatccagaataatrgatccagaaaagctattcttattttagagagtttttggcgtattacttttttttttaaagaaatgttgtattcagttgctaatgcccaggataaaaattagatctatggttctcaaactccagggggcctcaaaatttcctggaaaacttactaaaacgcacagactcccccaccccgagtttcagatccagtaagtctgagatgaggcctgagaatctgcacttctaacaaattaccagtgatgccgatactctggttcagggttcacactttgagaaccacttgattaggttgactgt

SEQ ID NO: 10 - DNA, 601 bp, Homo sapiens
ccaagatgttagacatgctactgttattattatgactttaaattgtgaattgaattgggtggtattgttagtgaaacagggaaccttattttatattacattaatggaattgtaactcacttacttcatggttatagaagtgtccattaatagaggctctatttttctgtctttcttttctgtccatcatcagaggcttcatcctttttctcaccagagctgcttcactcatggttctatagagcactggggagtctggttcatcctttgcatgtggcttcagggtgttgctgtgataagrtaaatagtctgggaagaatagattataagctcatatcattcagctgcctgtgtcctagcatcctgagtggatttgtgtatgctttcattaattttaggaatattgctattttctggctgtgtaaaagcattatagtccactgcttagctgcatatttacttattaaatgttgcctgagactgactcagcaaattctgccagtatctcatataaaattcttatgtctcattcttatgtttcataagaaatgatcacaatgatcatgtttggggtttgggcattcctttaatcttaatttat

SEQ ID NO: 11 - DNA, 8812 bp, Homo sapiens
tataaactccttctgaacagtttcatccacatgcatgtttaagtcaccagataagtgctggttactcctaaatataaagctcaaggtcttacctttctctttgtttccaatcatggattcttaattgtttcagtttatggaaccctgtctgctcttcagaatttttgaggaccctaaaaaacttttgcttatgtggattatatttattaatgtttaccatattagaaattaaaactgagaaaagtttcaatatttacatattaatgtatttaaaataataataaacttgttacatgttaatcttacaacatattttgtgaaaaaaaaactgttttccaaaacaacaacagagaaaagtgtactaaaaaaagaccattgtcttatggtttgaaaatcttaatatctagcttaatagaaagtatctagattttcatatccattcctgcatgcaatctgtaatagtgagaaaaactggcctcatatacatatgtacttggaaagaggaatattttaattgttcctcctttttaagattttcagataattatggatattcttttttgatattacaccaaaactaaaaagtagtagttccttaagggttagttgcaatgtgaaacctgaaaccacatcagtgaacttttcatactctattataataaaatatgctggtctattttgaaatttgaatcgatcttttacccatgcaagattttgtgacatgcattggtcatttggaagacattggtttactgagttatgcagatgttccgaatgttgacagatttttcacatcatatgaaaaaataacatttgttaatatggcgactgatctcaccagacaagtgttaaagtataggtaaacctttccaaattctattttttaattgaaaactcaattttcacatgagcaaaacatgcaaaggaaacactgcatgtttcctttgaagtgacaggttcattttgttcatttttgagaaaatgtcagccaaagatacaaaccatagtttacctgtctgttgttcttccaaataaaaatggcattccataaaaagtcgccagctcaaatggcgattcaaacaattttcacaactgttattccttgcgataagcattatagactgatatgcagcataaatgactcatgaaatttctagaaatacaattttaaaagcataaacatatgaaatatgagaagataaaccagtcaaaagttgtacataacttgatattgaaaactatgaactattattgagagaaactaaagattattttaataaataaagagagaatctatgatcttaggtcagaagactaagtattatttagattttaattctccccaaactgacatataatattaactcaaactcaaaatatcagctagaggaaagaggtggagcaagaaggccaaatagatggctctaccaattgtttccccatgtgggaataccaagtttaacaactatctacacaaagggaacaccttcataggaaccaaaaatcgagtgagcactcacagcgcttggttttgaattcctatcactgaaagaggcactaaagagggtgggaaagacagtcttgaattcctgatgccacacctcctctattccccagcaacaaccacttctatgcacttgggagagggatggggtagcgattgtgagcctttccattgaactcagtgctgccctgtcacagtggaaagcagaacggggctgtactcagcttatgcctgcacgtggagcaaacattccgggcaggccctcgccagaggggaattgccatcccagcagtcagagcttgaattccagtaagtgacataaacacaagctggagtgctctggggccctaagtgaacttagtgggccgtccaggacccaaagactgcaattcctaggcaagttctaagtgctcttgttcctcagggaagaacaagcagccatacccatatagcatgttgtgggcttttgagactcagatgtgcgggcttgagataacaaccagcacattcccagctatgctggctatgagaagtgactgctaaagcttgaggaaagcagagggaaaagtaaaggaggggacttcgtcttggaccttaggtaccagctcagccatagtaggatagcgcaccaagtgggttcttaggggttcctgattacaggccttggtcttagactgaatttctggacctgtcctaagccagaggggagccgactgccctaaagggtgagtcccaggcctggcaacatttaatacaagttaactgaagagccccttccttgggccttaacggaatgtgagtagtagcctggcagtactccatgtgggtctgtggtggtggtggcaatggggtgaagctccttggcctgtgcgaagtggagggaagagtgggaaggaccgtggcatgtgatttgagtgccagctcagctgcagtagaataaaacatcaggtagatttctaagcatttgaatccagtccttagctcccagaaagcatctctgtacctctctggggtcttggggaactcagcaccctgaaaggaaggaaacgaggatggctggcttcatcacctgctgactctagcgtcctgggaccttgcacaaacataggcagtaactaggagaaagagagatatgtgacctttcagagaattcaaaatagccattttgaggaaactcaaagaaattcaagacaacacagagaagtcatacagaattctaccagataaatttaacaaagagattgaaataattgaaaataatcaaacaaattctggagttaaaaaatgcaactgatatactgaagaatgcaccagaatctctaaatagcacaatttatcaaggagaagaaaagattagtgacaggccatttgaaaatacagagaggagacaaaagaaaaaaaaatagaaaagaatgaaacatgcctacaagatctaggaaatagcttcaaaagggcaaatataagagttatgggcattaaggagaagatagaaaaagaaatagaagtagaaattttgtgaaaagggaaaataacagagaatttccaaaatatagagaaagataccaataagaaggttacacactaccaagtggatttaacccaaagatgactacttcaaggcatttaatcaaactcccaaaggccaaggataaagaaaagatcctaaaagaggatacacacacacacacacacacacacacacacacacacacacacaccatatacaatgccgcagacagcagacttttcagcagaaaccttacaggccaggcaagagtggcatgacatatttaaagtgctaaagaaaagaaattttactctagaatagtatatccagtgaaaatatccttaaaacatgaaggagaaataaagacattcccagacaaacacaagctgagggatttcatcaacaccagacctgtcctacaagaaatgctaaagagactactgcaatcagaaagaaaaagatgttaattaggaagaaaaacatcaccctaaggtataaaactcactggcaatagtaagtatagggaaaaacttagactatcataacactgtaactgtggcgtgtaaaccactcttaagtagaaagactaaaagatgaactaataaaaaataataactacaaaaactttttaagacagaaacagtacaaagatcactgaaacaataaaattttttaaatcaggtgatgaaattaaagtgtagagtttttcttagtttacttcttgcatctttgataaatcgtttacacaagtactataaagctgttatcagcttaaaataataggaaataagatagtatttgcaagcctcatggtaaccttgaatcaaaaaaacatatagcagatacacaaaaaataaaaagcaagaaattaaattataccactgggaaaattaccttcagtaaaaggaagacaggaaggaaagaaagaaagaagaagagaccgtaacaccatcagaaaatgaacaagaaaattataagagtaataccttacttatccgtaataacattgaatgtaaatagactaaactctccaatcaaaagacatagagtggatgaatggatagaaaagtacgacccaatgatttgttgtctgcaacaaacacaaactataaagatacaaatagactgaaaacaaaaggatggaaaaagatattccacatcaatgaaaaccaaaaaagaggagtagctatacatacatatatatgtataaatatatatgaaatatatatakagagagaaatccactttttttttgatttgggtcttctttctttttttcttagtctgtctaatgattcgttaattgtgtttatttattcaaaaaaacaactttttctgccattaattttttgtatttttttcatttaaaatttatttccactctgatctttattatttctcttctattagtttatatatatatatatattttatgtttatatatatatatatttgatatatatatatgtttcaagacaaaaactctaagagccaaaaatcactatataatgataaaggagtcaattcagcaacgggatataacaattgtagatacatgtgcacccaccactgtagcacccagatataggaagcaaatattaatagagctaatgagagagaccgatcacaataaaataataggtggatacttcaacgctctactttgagcactggaataatcttccagacagtaaatcaataaagaaacatctgacttaatctgcactataaaccaaatagacctaatagatatttacagagcatttcatccaatggctacagaatatacattcttttcctcaacacgtggattatactcaacaatagaccatatgttaagtcacaaaacaagtcttaaaacattaaaaaatcttgaagtgatattcatcatcttctctgacaacagtggaaacaaaccctagaaacaaaaacaaatgagcaattttgggaactgtacaaacacacagaaattaaatgatatgctcctgaataaccagtgggtcaatgaagagattaaggaggaaatttaaaaatgtcttgaaacaaatgacaatggaaacacaatgtaacaaaactaatgggatacagaaaaagtaatactaagagggaagtttatagctatgagtgcctacagcaaaaaacaagaaaaacttcaaataaataacctaacaatacatcttaaagaactacaaaaacaggagcaaatcaaaccccaaactagcagaacagaaataataaagatcagagtggaaataaattttaaatgaaaaaatacaaaaaattaatggaacaaaaagttgggtttttgaatacataaatgaaattaacgaatctttaaccatactaagaaaaaaagatggaagacccaaatgaataaaataagagattaaaaaaggagatattacaactgatgctgcagaaatttaaagaatcattagtggctattatgagcaactacatgccaataaattagaaaaatcttaaaaataaataaataaatttctaggtacataaaatctaacaagtttgaacaatgaagaaatccaaaacctgaacagaccaataacaaacaatgagatcaaagccataataaagtttcccagcaaagaaaagcccaaaactcaaaggcctccctgtttaattccaacatttaaagaaataataccaatcctactttaattactctgaaaagttgagcaggagggaatacttccaaactcattctatgaggccagtattactctgataccaaaaccaaacacacacacacacactcacacacacacacagagagagagagagagaaaactacaggccaatatctttgttgaatattgatgcaggatatcctcaacaaaatactagcaaactgaattcaacaatgcatttaaaagatcattcatgatggccaagtgggttttttccagagatacaatgatagtttaacatacacaaatcaatcgaaatgatacatcatgtcaacagaatgaaagacaaatactatatgaccatttcaaatgatactgggaaagcatttgataaaattcaacatccctcgtgataaaaccctcaaaaaactgaacatagaaggaacatatctcaattcaataaaagccatattaaagactcacagctagcatcatactaaatggggaaaagctgaaagccttttatctgagaactggaacataacaaggatgcccgcttttaccactgttattcgacatagtactgcaagtcccagatggagtatcagtgatgagaaagaaatgaagagcgtccaaattagaaagaaagaagtcaaattatcccttttttagatgttataatcttatatttgaagaaaactaaagactcaaccaaaaatgattagaactaaaaaacaaattcattaaagttccaggatacaatattaacatacaaaaattagtagcatttctatatgcttacagcacaatcaaaaaaaaagctacaaagtaatccaatttataatagctacaaataaaataaagtacctaggaattaaccaaataagtgaaagttatttacaatgaaactataaaacattgttgcaaaagattgaaatgacacaaaaaatagaaagtgttccatgttcacggattagaagaatcaatattgttaaaatgttcatgctacccaaagcagtctacagattcaatacaatcactttcaaaataccaacaacattctttacagaaatagaaaatacagctctaggacttatatggaagaatcacattatctgactgtaaattatactacagtggtgtagtaaccaaaatcgaatggtactggcataaaaacagacaaatagaccatagaataaagaatcccaaaataaactcagacatctacagtgaactcctttttgataagggttccaagaacatacattggggaaagaacagtctcttcattaaatggtgctgggaaaattggatacctatatgcagaagaaagaaactacactcctatctcttaccatatacacaaatcaaatcaaaatacagatgtaaactaagacctcaaactattaaactacttaagaaaacattggagaaactcttcaggacatgggtttgggcaaagatttcttgagtaataccctacaagcatggacaaccaaagcaaatatgaacaaatggaataacatctagttaaaatgcttctgcacagaaaaaagcaaatcaacaaagtgaagagaatgcacagaatgggagaaaatatttgtgaactacccatctgacaagcgattagtaaccaggatatatatggagctcaaacaactccctaggaaaaaagtcagataatctgttttaaaaatgggaaaaagatgtgaatagatatttgtcaaaagaagacatacaaatgtcaaacaagcttaagaaaatgtgctcaacatcgttgatcatcatataaatgcaaatcaaaaccacaacgagatatcatctaacctcagttaaaatgatttttatccaaaggataggcaataacaaatgctggcaaggatgtgaagaaaaggaactcctcttggtgggaacgtaaattagtacaaacacaatggagaacactgttggtgggaatgtaaattagtacagccactatgaagaaaagtttgcagattcctcaaaaaactaaaactagcactaccctatgacccagcaatcccacagctaggtatataaccaaaaggaagaaaatcagtgttagtgttatctgcatgcccatgtttattgcagcactatccacaatagccaagatttgaatgcaacctaagtgtccatcaacagatgcatggataagggaaatgtggtacatatacacaatggaatactattcagccataaaaaagaatgagatcctgtcatcctgtcattgcaataatatggatggaactggaggtcattatgttatttaaaataagtcagacagggaaagtcaaacttcacgtgttctcacttatttgtgaaaactaaaaattaaaacaattgaactcatggagatggagtagaagaagggatatcagagaataggaagggtagtcagtggggttggtgggaacagagtgatggttagtgggtacaaaataagtcagagggaatgaataagacccgatgtttgctagcacaacagggtgactatagtcaaaagttattttaaaataactaagagagtataattgtatttttttaacatagaggataaatgcttgagggaatggataccccatttgcccgatgtgattactgtgtattgcatgcctgtatcaaaatatctcatgtaaccccaaaatatacacaccctactatgtactcacaaaaaattaaaaattatatatatgttcatatatgacacatatatcagctaaatttttaagtaaaaaatggcaagctaatcctaaaatttatatggaaattcaaagaacctagaagagcaaaacaactttgcaaaataataataatattagataattaatacaacctgactgcaaggtatattataaaggaagcgtaatcaatgttatgtggtattgagataatgttaggcaaatgtatccatgaaacaggataattttctggaaaagatctacatacaaatgaacagattattttctacaaaagtagcaaaattcagttgagaaagtgtggtatagtaaattttattcacacaatctaaattaagcctccataaagtttatttaaacagaagaaataggtcatagtgcccccaaaatttgggaactactcaagattcctgggattgttggaccaacatgaaat

SEQ ID NO: 12 - DNA, 1127 bp, Homo sapiens
agaaagggtagaactgaatgaaggtctactcaggctagctttctcttttctggcagcatcatcaggcacattgaatttcacatttttttttattttttagttttttgagacagagccttgttctgtcacacaggctggagtgcagtggcatgatcttggctcactgcagcctctgcttcctgggttcaagaaattctcctgcctcagcctcttgagtagctgagattacagtcgcacgccatcatgcctggctaatttttgtatttttaatagagagggggtgtcgccatgttggtcaggctggacttgaactcttgacctcaggtaatacacctgccttggcctcccaaagtgctaggattacaggcatgagccaccgtgcccagctgaacttcacatatttttaaatgtaaaaaacatttttaaatggaagtcagtcatgattttcttcacaaacatttcatttcactgcaattctagcttttctccccattaagaatagagagtgtgttagtccattttgtgtttctataaaggaataccagagactgggtaatttgtttttaaaaaagaagtttatttgtctcatggctctgcaggctgtagaagcatggcatcagcatctgctttgcttcaggggaggcctcaggaagcttttactcatggaagaaggtgaagggaaagcaagtgtgtcacatgaagagagagggtgcgagagagagagagagagtggggagatgccacactcttttatacaacaagatctcagctgaacttaytaccactcattaccttgaggaaagcaccaagccattcatgaaggatccatccccatggcccagcacctcccactaggccccatcaccaacactggggaatcacatttcaacatgagatttggagtgaacaaatatcccagccatatcaaaagggttattttcaattgttattgtgaacgtgttctcttctgtcttgtcaaagctcattaacactgcacactcaatctgcagacaaaatggtggacaagattgttgaatgggtcagtgaatatccgtccaaggctgaggcacttctgtagggctcaacaagctgtggcaatcaacaacagtccaaccgagcaacacaca

SEQ ID NO: 13 - DNA, 831 bp, Homo sapiens
tttattatgaaacgtcactttttaaaagatataatgtttcacatattttaaatcgatttatttggagacaagtcttttgtcgtaggatcaagtatgcgtctaagaggaaaatagacggtaggtatatggaaagtgtgttttctgtggcttttctggttggcttacttgctctgtttgctctaaagactattaggaggaaartgataactaagaaaacagaaagtttgttcaaaatggtgtggaggaaaaaggaaggaaagaaaatttttatctgaagaatgagagcccctttaaattatcaggccccaaaaggcactgaatgaaacagcagtcacatcactcccccttgaactaaataattatctcttgaagccacttgccatgtgggctctagactaactgatgccaagtagccatcaaatgccatatgccagacactataacactatatcctgcagtcccacagtgcatggccaatcattaagcaatgttattctctgaaaaccagtgagaattcctgtcaaagaactttacatcagaccattccttgttccctttgcctttagaaacctgcttacaacaaaagctaacagagcactccccagtgcaactgggaagagcgtctcaacagctgtcttccctttggctcaagtaaattctttaaattatattttgtgcctcagcctcctccttttaggtcaacaaggacatcaactaatacctccaggtgtgtgcagctttagtattcacataaataagattttttaaagccttaatttttagcaaccgtcactaatcattaagcccattcacattttctaaatcgagtgt

SEQ ID NO: 14 - DNA, 601 bp, Homo sapiens
tatttatataaataatatacgcttatatatgtatatttgactaaaaatttaacttaatttggaacagcgttgctactgaaaatgaagttgatatagtgcttttatcccacaggtttgtttacatattaggggtttagcaaatagattttggtgttaaaggaaagggtgccctcttcaggcaatatgtctcatattgctaaagattttgactagaaaaatgtggacttaaaaaaatactaaaaactcatagaatcacaccacgaagaggcattttgtttggtgttctgtgtctattcttacycagtacaaagcattttatttttagaaaactccaggtaaggatttttaaatttccatttcataatgtgacttagtttagttttttttaaatctctatttcctttttatcttaatatctatttcttttcaacatatgagataccttaggctcctttcttggaagatttaaggtacaactacagaaatgcttgtcatgggatacctcttttggcctgtattaacagcagtgatgggaaagcattccagaattcgatttcttttcaaaaaggaaaaacctactaagatctaagatttaatgggg

SEQ ID NO: 15 - DNA, 3900 bp, Homo sapiens
aaacattgaatggttaattttatgtaacattaatggtatatctaatacacagatacaagagattgtaatgaacaagagaggtagaatttttaaaaaggctcagaaaagtaaattaacaaccccaaatcacagaatcaatctgatggataaacattaaatattagttttattaatggtaaaacctggattgaggctttgcatcaaaaatarcacattgtccacatacattgacttccacctcttctaaaaccacactaaaatggaaataaagggatatatttggaagacataaatccacaaggacaaagagaatgagacgaggtgggaaaccacagcaaattgtggagggtggaaagtggttggaagagtggggactgtcatggcagccttgagagaactgaatcccacactggctgtgggaaagccaagagcaactagttcaaaggtagactccctctaccacacacagattcaggaattgatagacccagatacttctgcaagtcaaggtgaaggtggcttagaccagaaagattgaacaaaaatctgtttaagaagcagttagatctctagactccttgtaccccatgttagtggtgattgcccgtttactacacacaccagcagaaggctggagatttattctctagcatttacattgaggatttcttgactgaggaaccctagatatggattaagttcaggacaccaaagtgaagataggaggaataaataaaaattgatatactatgaatattgataccaccccatgcttcttgttgatctattttcctggaatgctggtagaaccttatgagattgtaacctgcccataaagaaagaacaagaactattgatgttgacagttcccccagtgaaatgctcagccaagtcacctaacagtgaagcccatatagcacgcagaacttctgatcaactttttgtcccctacccttaaatacgagatgacagccgaaagtcaccacattttgagcaaaccctccaatatgaaagacaaaggccaaaataaagaaaccacaagaagaaaataatctatatgaaagagagatttatgcatggaaaagataatgtgaaaaaaacacaaaattaatatcttaagaaagatgaggaaaaattgtgcttttcttatagttaaatatgagattatatgtatataagaatattattatcttataattaaaaataagagaagatttagggaatagaaaatagttcatgaaaattaaaaacataatagtaaaaatgaaaatttccaaagcatgtttagaagaagaaaaagcgcaggaagtcttcctaaaagcagaacaaaaaaaattaaaaaataaagagaaaatttttttttgacaggatcttgctctgttgcccaggctggagtgcagtggcataaacacagctcactgcagcctcaacctcttgacctcaagcaatcctcccacttcagcctcctgagtagctgggagcacagccatttgccactaggctcagctaatttttaaaaattctttgtagagatgggggtcccaccatcttgcccaggctggtctcaaactcctgggctcaagcgatcctcctgccctagtctcccgaagtgctgggattacaggcatgagccaccacacccagcctagaaaagtgttttataaatgagaaatcaaaggagtaatttaggaattacagaaaaggagagcagagaaagtggagggaaagaatcatcaaagaaataattcaagaaaaattcccagatgtaaatttccaaattaaaagggccctctgagtacacagtgaaatacatctaagacccatattaagacacatgatagtgaaatttcagagcattcttgccaaatgagggagtgaacccaagaagatgacatgggatctgagaaacaggtgattcataagagaaaaatgaaaagtatctctagaaccatgttacaggaagacattgaaatgacctctgcgcagctgacctatcagtcaatcccaatggagcaggtcagaaagcaccaggagagacagcttcataaagttcagaatggtagaacacctaatggatttgaaagcactgagaggagattgactcaactgtaggaaaagttgggaataaattagcattaggtattagaaatgtaggccgtcttttaaaaaggcaattatataagttctagagaaataaattgtcaggaaaggaaaataagtaatatatgatttagctgccaacagtgtttccattttacatggccataaaatagaaacactgaatattgatctaaccaaacttacaacatagtgggagggtgagacattgggaggagtgtttgtgtgtctgtgtctagacgtattttttaaaatgtaaaatcaggcctggcactttgattcacacctgtaatcccagcactttgggaggccgaggtagggggaacgctttgagcccaggagtttgggaccagcctgggcaagacagggagaccccatctctacaaaattttttgtttgtttgtttttgagatggagtttcactcttgttgcccaggctggagcacaatggcatgatctcggcctattacaacctctgcctcctgggttcaagcgattctcctgcctcagcctccttagtagctgggattacaggcatgcaccaccacacccggctaattttgtatttttagtagagacgatgttggtcaggctggtcttgaactcctaacctcaggtgatctgcccacctcggcctcccaaagtgctgggattacaggtgtgagtccccacgccccgtcaaaaaaaatttttttttaattagctgggcatctggtccccgctacttgggaggctgaggtgggaggatcacttaaacctgggaggttgaggcttcagtaaggcgtgattgtgccacttcactccagcctaggtgatagagtgagaccctgtctcaaaaaataaaaataaaaagtaaaatcatctctacagtgggaaataagtgtttaaaagtaaaacaaacaaacaaacaaacaaacaccaaggcatatctatataggaatgttcttcagaactattgagggaaatacaaaagaattggttgaaaagaatgaaatgtagttgcattattggaaatggtgaagttgtagagctgccatttttcttaacaagccttttagaattatgtaactctttaaattatggacgtgtataactttcattcaaaaatataaaacaaaaaaatctgtattgaagaagaggctgtgaggtgaagtcaaatgggctgcacattaatttggcccacttggtctttattcaagagttgaagtctggggcaccccaaagtttagaggtacctgagaaggcacagccagaactgtaggagaaaaacagagagtgtggtgtcttggaagccaagtgggtcgagtgtttcaaggacaaattgtcagctttctcaaatgcttccaagaagaaagtaagaaagttcataaattggccaggcacagtggctcacgcctgtaatcccagcactttgggaggtccaggcgggcggatcacaaggtcaggagattgagatcatcctggccaacgtggtgaaaccctgtctctactaaaaatacaaaaattagctgggtgtggtggtgcatgcctgtagtctcagctactcaggaggctgaggcaggagaattgcttgaacctgggaggtggaggttgcgatgagctgagatcgggccactgcgcttcagcctggagataaagcgagactccatctcaaaaaaaaaacaaaaaacaagaaacaaaaaagaaagttcagaaattaccataggaatgaagaatacacaaatcccaggtattttcttgggatttccagccgacagaaaaaaagccattggcttcctagtt

SEQ ID NO: 16 - DNA, 888 bp, Homo sapiens
gggtgcgctgcagggggtgggagtgagcgaggaggaattgcggggctagggtgggttgctggaacctggcacgtgtgagaagggtttgccatcgtctctgatgcatccaggctacagccagccgtgagttcctgctgcgcgccaggcacatgtcagatgctgggataccaggaagtgcacccagataggggcctgaagactatgtagaaactcagagggtcaagaacagccagaaaaaaatcttgaaaaagaaaagcaaagtaggaagaccagtggaacataagatagagaccagaaacaaaaatacacgtgtatggacaatttatttcagaggcggggctgcagagcacaggaaggaactatttttggttaatggtgctcggtcaamgggatatccacgtagggaaaagaaaaaagggaacttggccccctacctcccatcacacacagctctgaactccagatcaattgtatctctaagtgcgaaaggtagtgtagcaggaggagccgcagacaaaactcctcagacaccggattaaagaaggaagaggtttttattcggccgggagcgtcggcagactcgcgtcttaagagccgagctccctgaaaaagaaattcctagccattttaagggcttacaacgctaaggggtctgtctacgtgaaagggtcatgatagatcaagtaagcgtgaggaacgtgactgggggctacatacatcagctaacagaacaagaagttttacagtgctttctcatacgatgtctggaatttacggataacgccagtagttttggtcaggggttaatattattattattattttaaccaccagggccaggtggtggcgccaaggtcgtctagctatttatcttacttctgttttttt

SEQ ID NO: 17 - DNA, 4552 bp, Homo sapiens
ggaacagtaaaatacccacgttaaaagctctttctctgtgcctccttagcaccctctgatccttcatagagcccagcaaaaatcaaccatactataagcatccttcacctgccttgcagctaagctgaaggacaatgaggacaatgatgccgtcctttctaccccatgtcccaagctcccagagcagtgcttggtaaaaaaaaaagggtacttactaaaatgttggctgaataaaaggaagactttgaaaaattaagttattttgtacccctatagtgtgaattctttttattgaaatttaatacacttacagaaaaaaatgtacatattataattgttcatagagctcagtgaattttcacaaactgaacaaatctgtaaaccacacccagattgagaaagggaacatgacaaacaccctgcaagtctcccttgtgccctgtgctggggggtgccaacaccaccacctcctagtctgatgatgtactaggagattcacagactcagcctgtagtcatactcatggctatgttttactacagcgaaaggctaggaagcaaaatcagcaaaggaaaaaagcgcatggacaaagtccagggaaaccaggtgtaagcctcccacagtcctctcccagcagagtcacataggacacaattcctccagcaatcagttgtgacaacccatgtgcaatgttgtatacaagggaagctcattacagactcagtgttcagggttctcattgggagctgctcacctcatcctctgcctaacacatatgaaaatctgactcccagaaggaaagcagatgttgggcataacccacattgtttgtacgaacagtttaggcacagtgagccactcttatcatttagggagtaatgagaaccctcctgaaatccaagctttcagatgccagccaagggccagccttgcaaacaggcctgctttctcatctcttttcacacagtccatctccttattgaggcttctttacagcaaaattattattagtaggaccacatgcaaaagggtgagaccaacctcaaatatggacctcagttcttcctggtaggggtcctggacttaaccagctaaaataaatcccattaaccataaagtctatcttaggaagccaggtggcttcctccttaattcctttttgttgttgttgttgagatggagtcttgctctgtcacccaggctggagtgcagtggcgtgatcttggctcactgcagcctccacctcctgggttcaagcgattctcctgcctcagcctcccgagtagctgggactacaggtgcacaccaccacacccagctaatttttttatttttgtagggactacaggtgcacaccaccacacccagctaatttttttatttttgtatttttacttttcaccatgtcggtcaggctggtctcaaactcctgacctcaggtgatctgcccacctcagcctcccaaagtgccgggattacaggcatgagccaccgtgcccagcccttaattgctgtctgaaccttttttttttttttcaacctgacctgaggcatcaattcaggcacagtataactctgaccagtaagcggaagctaacccaaatgctttccagggctgaggggacatcatgagagttgtggtcatgtacagaagcacagtcccaggggcacaggtggtacccctgtaaacaaagaatttgcaatattagggcacctcaagcaggacatccattaggctgtacaggtgaacagacctcctaccaggggaactcctgcccaagtgtttccagtcccatctgaaaagttcccagagtccttggcttccaagggactgcctgaaggcagtgagtcccaaacagttttgcttgttcaggatacaagttgatccatctcaygagcatggatgacatttgtgggagatcttcatggaaagcatagaagctagacccactctgttgtcagtctgaactcaaagcactcacagcacatggaaaggcagcactgggaattttccttcagcaagaggaggcttccagggatcaagcctccccaggtgccaggctttaattattttctccaatattacaaaaacagtcgtgccctgttcagcaatcataacattgcattttttcaaccatctcttactctcagctaggcctttcctctggaagggttgtatgtaagaggcacaaaagcatttttgtagataaactttttttttttttttttgagatatggtctggctctgttgcccaggctagagtgcggtggcacgattttggctcactgcaacctccgcctcctgggctcaagccatcctcctacctcagcctcttaagtagctggaaccaaaggcacatgccatcatgtccagctaattttttgcattttttgtagacacaggatttcaccatgttgccaggctggtcttgaactcctgaactcaagcaatctgcccacctcggcctcccaaagtgctgggatcatatgcatccatcactgcgcctggccaacatttttttttttaactatcatcctagagttcaagtagataaacgtttttatgcaacttaaccagaaagacattcagaaattggtaattatgtgtggagggttgaattatgtccccacaaaaattcatgttaaagccctaaccatttacgtgtccttaaaaatgtgagtatatttgaagatagggcctgtataggtaattaagttaaaatgggatgtctaggatgggcccttaatccaacctgagtggtatccttattaaaagagaagattaggacacagacacccagacagagggaccatacaaggacacagtaagaaggcagccatctgcaagccaagcagagaggcctcagaagaatctaaagctgtcgacaccttgtcctggactttcagcctccagaactgcaggaaaataagtttctattgtgtaagtcactcagtttgtggtactttgttatggtatccttagcaaactaataaaggatgaaaccctgaatttttaaaaatttcaaaatgtgtttacataatcttctaccccttttgtcctgattattcaatgagcagactagaaaagcctaatacctttctggggacagctacgttttaataatccacctaccttactaagctgtgtgtgctgggagaaaagtgagggaagtagagtcaggctgtcagtccactgttgcaagctgcaactaatcccaaaagctgaatgcaacaagtcaacagcagtgagagaagccccagagaaggtttgagtctggtctgtcaaaagcaggaggagaggtcatagtccttgcgatgtgcccttccccagcttggagcgtttggcaaatattagaagttaggaatgcaattagcctctgtattagagacaaagagcctatcagcagcttgatcttagatggtcccattagagaataagcccttttcagccaggttccaggacgttttcatagtttagcgctccacggagggggtccttaatctccaacggccccaacgtctgagtcctggtcattgagcctgtgcctgctttcagttcagcatagctcgtgttgaggctaaaacagctcaaggcagacagtagcaagtttcagcttagagtcagggccaggccattaggatctgagaatttgagattctcaacaagcagctgctttttttcagcagcagtgaaagcatgctgcatggccagacagctgtggtccaagtgacatgcaaagctgcctttttatcttgttggtcttagagtccaattgacccactcaaaaaaaatgcatattagactagacaatcatcagcctctaaggggagacatttgattttgacaagggttcattagcaatttaactgtattttcattttccaaacagattgcttctctttacatgttgtctccctctttcccgaggctccagtaagcacgaatcacagcaaaaataatcagttacagcctgttctggttccaatatccgaagaatttaaattccaacctatccactagtggcaaaaaactagaaagctgtatcccactaacacctttcatattttatttttgagtagctttatttgattagggtgatccctctagcatttatgaaattaacaagcataaaactttttttttaagagatgagtagctgggactacaggcgcacgccactgcgtccagctaaggtatgttttttaagcaatgaaatcaaccagtaaaaacaagttcaaatatttttcattcacacactcaaggcctgctgagcatccactgcatgtgttttacttgttccattagaaaggtttattaaaaaatggcggggcacagtggctcatgactgtaatcccagcacttagggaggccaagatcgcttgagcccaggagttcaagactagtctgggcaacatggtaaaaccctgtctctatttaaaaaataaacaaatacaagcaagtttttttttaaaaaatgatatattaaagaaaatcctcctaattctcttatgtcttttcatagctgtttccatgttctcatattcccatcct

SEQ ID NO: 18 - DNA, 601 bp, Homo sapiens
ttgttctaaaaatgtcttttcagtttttcaagttgacgaagcacttaagccccaggttcttaaaaattatttgaagtaaaacttaactttcatctattctgtctggagaatgaattaaggcaatatttcctttacgtcatgattgaaagtcctatttgcaaacttctgctattatgttttagtaaaaatattttatagaagagttttaggaactctgatttagttttcaaaggtaactcctagaaaagtacttttgaaaaattataatttttactctatatttcagaagcagtaaggagaygctatagattgatagctctttatctccactgacattcagtgatggagcttctttcttcatttctagctatgaagattaagcacctcataaagttgcagtataaatacctagaaattcatctgtgcctttttctgcatgatattatgttgacaagctggtatcaagcaacctaacacacttaaagattaacattaaaactgatataaggttttacatttaaatctttaatccatcttgagttaattttttgtatatggtgatacataggggtccagtttcagtcctctgcatatgatagcc

SEQ ID NO: 19 - DNA, 1416 bp, Homo sapiens
aatgcataaaaagtattagaactctccaaaagttcctacgccatttacacctccagtattgaaaatgatacaaagatgaaatgcatagtaagttgtaaaaaataatacaatttaaaatagtaaaaaataaaactaagaaacaaaaccaaaaataaaatttgacatatgaaaaagtatattattacaaggatagattgtgggcaattgcacagagataatccctaagaactggcagactttcacggtcattaactatattttgaagtctttcatcacaatgaatagttgcgtttttcttttagggcatgactctcttcagataatatgtttagattcatttccaatgtggcactgctctttatgaaattcttctatgattctatgtatactgatatgagcatttcctattaaagctttctaccttctgtgccatgcttctgtgttgtttagggtttacagaaatccatttcacatgccctcatatacagaacacaagtttggcagaaacaatgctggtgatcgaacagcaatgccattgtgaacattacatcttcttatcctactgtgcacatagtaatttttgaaacagtcagtaagctttactggcttcttcaggcaaattcagctttaattcattaaaacctcctgaaatgtcataagtaggaaggtatatgatgcatttttaaactgaagttttctttgttgctgtatgcagtggctaatccactcatttgacttttccaccaaatgcaaataaagacaataacaagataatagttttgcctaacatgacgtaactatcctgcatacaactgaaaatgcactaatcccttccccagactttggctttccggatttcaacattcgggattttaatcttttggggttgtgaatttcacaattttagacattaggaattttgatctttcaggatttaaacttttggaatttcatcattcaggattatggcatttgaaattgtgtcttccaggattatgatycaaaggcaaggcactgatgtaaattcctgtctagagctgaagggctgagaactaggagcatcaaaggcaggagaagataaatgtcgcaactcaagaagttaggcaaagttaattcaaacttcttccaccttcttgctctattcaggccttcaaagggttagatgatgcccactcaacatcggagagggccgtctgctttattagtccaccaattcaaatgtttatctcttctagaaacaccatcacagacacacccagatattttaacctggtatttggccccctggggcccagacaatttggcatataaaattaagtatcacagtgcggatagtatatttacttaaaaatgttgtgaatattagcttgttaattaaaaatctatggattttactttttaaaaatctggatttcc

SEQ ID NO: 20 - DNA, 1250 bp, Homo sapiens
agctttggtaatagatcccagattaatttttagcccaagcaacaggaaattcctcttaggccttagggccttaccctgcagaaatatttttgttggtgcctatgttagtcaggattgtccagagaaacagaatcaataggagatagatagatagatagatagatagatagatagatagatagatagatagatgatagatagatgtttgatagaaagacagataaaagagattactataaattattggctyctgcagttatggaggcagagaagtcccacattctgccacctgaaagctggagacccaggtaagctcttgatgtagttccaaggcctgagagcaagagagccaatggagagccaatcataatccagaaaggtacaagccctatcactagaatcctgaatgttgaaatcccaagagatcaaaatccccaaaatgtaattctagaaaaaataattttaaaattatctaaaatatttatttacatttttaaaggaggatttatctaagaaacataaaaacatgaatgaatacttcataggccactttatacgataaaataagcaataatatacatatgtttgtgagtataaacactcagatattctaacaacagtcacatgggtataacagttatgagcagatatactgtgttcataaagaggtcaaaaagggaaatgtataaacacatatcactgtggttggtaattgtgtgcacctagctttataactgaggtcatctgaaacaccatgacaaaccacctaagtcttttgatgggaccaatcaaaaccaccatttacaccatcccccaaagagctaagatcttgagaaattctatctttcacaatgtagatgtacagaaaggatatctctttatttattgaggaagtttcagcatttttatgtatatgcataatgtttaaatacagtccacattgtgataatgtacttcatggaatcaaatttgaaaatgcataaaaagtattagaactctccaaaagttcctacgccatttacacctccagtattgaaaatgatacaaagatgaaatgcatagtaagttgtaaaaaataatacaatttaaaatagtaaaaaataaaactaagaaacaaaaccaaaaataaaatttgacatatgaaaaagtatattattacaaggatagattgtgggcaattgcacagagataatccctaagaactggcagactttcacggtcattaactatattttgaagtctttcatcac

SEQ ID NO: 21 - DNA, 1029 bp, Homo sapiens
caacaaacagactgccttggagggtgggaggtaggaaacaggaacagcaggagactgagctccataaaatgtaattgtatttctactgctgcagacatagaaaattatgcctaaagaagccccagagtataaattggctaggaacattgtgttctttctaggcactgttttaagatggttagtgacaaataagaaggttcataagaaaggtggttgggtctgaaaacagatttggagactaattgatggtgggaatgttttgcctggaaaaaggaagacttgtagagggaagatagtaacagagtcactgtttccaaatatctgaagggctggcaggtggaaagatattaaatacattctatgtggctccagagggcagatctgtgagtatcggactacaaagtggcagatatcattagctctccataataacgggtcaaagtgaaaaaaaatgaacacttaaagagtgccttccaagggggtgagcttcccatccctggattcactcaagcacagagcacatggccacaattcttcaagaggatatctacatttggtcagtgcatgggccacttggatttgtggtgcctctctttacacatttgacccatttgatcattaccgccattttcaggttttacaagttctgtaacaaatagtggggggttttttgtgatcagtattgcaatgtttgggtgactgcctagtgaaacatattgttttcattgttaagttcatggaaataaaaggtaaaaccttttctttttaacttagaggaaaccacactgtatttatttttttctcattcccagcctacttcctgcttttcctgcagctcagcattgcatattgatggtccttgggctttgcttgcctcttcacatgtgttttatttttgcctgcaaagtatttttggtgatttgctttattgtgactgtaaagtattktttaatgagtttgaatgctgttaggcatattgctcattctctaggcctatacagttgccacgactgccttctacctaggctt

SEQ ID NO: 22 - DNA, 1650 bp, Homo sapiens
agttctcaagattattaagacttaggatgtgtcctcaaggagtttgtagtttgggcagggattgagatcttgccctactgtgtggccagagcataagatggcaggaaagggggctggggtccagacctaattcaaaggaaaggccggggttctggtatcctttcatcacttggcatttccaggtttcagaggaagaaaatyaagttaaaatcctgattccccatttctccttctggcatctctgattggcatcctttacaaggatggaatttcatgacttgtgtatttgtgtacttgtgaatcagtgaccctcttgttttggactttatgggagacttgcgtggggagagggaattgtgctcattcacctgggggggttggggtgatccccacttgtcaaaggtgtttgaaccagagcaacgccatcttgagtagcagctgggcaaaatgaggctgagacctgctgggttgcattcccaggaggttaggcattcttagtcacaggatgagacagaagatcagcacaagatacaggtcccaaagaccttgctgataaaagagaatgcagtaaagaagatggccaaaacccacccaaaccaagatggccacgaaagtgatctctggtcgtcctcactgttcattatatgttaattataatacattagcatgctaaaagacactcctaccatcaccatgacagtttacaaataccatggcaccttccggaagttaccctatatagtctaagaagggtagggactttcagttctgggaaatctctgcccttttcccaggaaactcatgagtaatccatccctttttagcatataataaagaaataactatagccgggtgtggtggctcatgcctgtaatcccagcactttgggaggctgaggcaggaggatcacttgaggtcaggagttcaagaccagcctggccaacatgatgaaaacctgtctctactaaaaatataaaaaattagcctgatgttgtggcgcacagctgtaatcccaactacttgggaggctgaggcaggagaattgctagaaaccaggaggcggaggttgcagtgagctgaattgcaccactgcactccagcttggacaacagagcgagactccgtctcaaaaaaaaaaaaaaaaaaccggaacaataagcatactcagtcaagcaactcatgccgccgctctgcctatgcagtatccattctttattcatttagtttcttttttttttttttgagacggagttttgcttttgttgctcaggttggagtgcaatggcgcgatcttggctcactgccacctccacctcctgggttccagcgattctcctgcctcagtctcctgagtagctgggattacaggcgtgagtcaccacgcccggctaattttgtatttttagtagagatggggtctctccatattgatcaggctggtctcgaactcctgacctaaggtgatccgcctcctcggcctcccaaagtgctggaattacaggagtgagccaccacgcccagcccgtttactttcttaataaacttgctttcactttactctataaactcgccccgaattctttcttctgcaaggtccaagaatcctctcttgggatctggattgggccccctttcaggtaacatactcat

SEQ ID NO: 23 - DNA, 1001 bp, Homo sapiens
taatcgtaagaaaagaaattcactcaagtttctcatccataaatggttacctgttcaaatctgaaacatgatatcaacctgatgaacatcaacctgatgattatcaaaaagctcatgattttataaatatatgcccaatcaccgtatcatcctgctgaggcttgggaggaggagggaaactgataggaacatgcagctcatgatatctcttgtgccaggaattatcaaagtcctttgtctctgacctaagagtttcatgtcttctgtcaatatctatgaaattgtggctaacttgttagcttgcaagtagagtaaaatctcagatctttcacagtttcatgacagtacaagattccccagtgggcctgctttgtgatgttctgagaatctttcctcaaggcccagacagagctcacttctcctattcttttaatgattttataaacaactaatttcctattttgatctaatttctgcttaaatactagggtggtttctgtytcctgtactgaaccctgaataataaacatgtccactataccatatcagactttcactttaaaccttcaaaatgccattcattattaagcactgttctcaacagggaaatatttaccacttaatcatcatttggaaacaattttgtttttgcttttttttaggtaacaatctagctctgtagcccaggatgcagtgcagtagtgcaatcgcagctcactgcaacctctgcctcctggattcaagcaattctcctgcctcagcctcttgagtcaggggggatcacaggcacacaccaccatgcccggctaatgtttgtatttttagtagagatgaagtttcaccatgttggccaagctggcctcgaactcctgggctcaagtgatctgcctgcctcagcctcccaaagtgctgggattaggggcatgagccaccacacccagccctggaaacaatatttttaattgtcccatcactgtcacagaacttgatctgatgtgtga

SEQ ID NO: 24 - DNA, 1262 bp, Homo sapiens
acactaaaactaaaaacatgttacgattgagacctggagtctcctacagataatttaaagagaaataagatatctttctagcaaaagttgagatcattcatgctcagagatgaagagacagaagagagaatctttcctatttcttttaaatgtaatgagtgtattaagatgatactcattcatgcatctcagtagcaccacttgggaaaattacttaatatctttccatttggtttcttcatctgtaaatgtaggaatgtttagcccagagggccattataaagatgaaaggaggtcattcatttggttatttaacaaatacttatttatttattgagtgtctattgtgttagatactgttctgagttctgagttctagggacaaagcattgaacaaggaggaccagggccctgcaatgagaaagttgacatgctaggggaagagacagacaaacaagcaaagtaacaagtacataagaaaacagtatgataagtatgrtaagaaataggaaacacagggctgggtgtaatggctcaaacctgtaatcccagcactttgggaggttgagccaggaggattgcttgaggccaggagttcaaaaccagcctaggcaaaatagcaagaccccatctctacaaaaacaaagaaaaagaagaagaaaagaaatagaaaacatagtggtgcagtgtgattgaatctcacctctgaggaggtgacataaaagctgaagcctgaatgatcaggggaaccagccccgtgaagatctaggaaaaggaaaagggcaatttaggcagaaagaagagcaaactgggctgggtgccgcagttcatgcctgtaatcccagcactttgggaggctgaggtgggtggattatttgaggttaggagttcgagatcagcctggccaacatggtggaaccctgtcactactaaaactacaaaaattagcctggcatggtggtaggcacctgtaaccccagctacttgggaggctgaggcaggagaatcgcttgaacctgggaggtggaggttgcagagagccgagattgcaccactgcactccagcctgggcaatagagcgagactcagtctcagaaaagaagaagaagaaaaaaaaagcaaactaggcacatacatgtttttggtttttttattgaagctatattattatttacagaaaagggaacagacaacatcccttcaaactctttcatataaattagctttcatcacattaagcagaaagcatataaatcgccatg

SEQ ID NO: 25 - DNA, 665 bp, Homo sapiens
gcaaattatgttttgatgcaataactcaaaacaatgtgtacctgtccacaaatttaacattacctttgggcccaaattgaaggacttgtggcagaatagctatccttagtgcccagacccctcatttgacacttatctacatacaaccatcatgagtttttcttcaccgtgcctgcatgtgttcatgtccttatgcctcattatcccaccttgatcataaactccttcaaggcaacggccatgcctgatcattcattttatgtccttctgtgttaacccaatgccttgtccatcatcagaacagggcaggggctggttgacagatgagtattcatgccaacaaaaacactcttccygtcgtacagtttaaagagcccatttgtctgttatgacatatcactcgctttggtgtttgttactggagtgcccaaccagtcagcgttgataggtcactggacacattatataatgtgctgaaattaactgaattaatattgtcaaaatggcttggcttttaaatgcaggcttctccttcaaagacacgcagcatggtctggactctgaaagacaccacttttgcataaaaaatacctgcttattatgactgttacagagaaccttattttagtcactgatcctgaaagtcactttaaaaaaaaaaaaaa

SEQ ID NO: 26 - DNA, 2115 bp, Homo sapiens
tatttatttataaagcacacaattgtttaaaaagatttattatgcatatggattatatataagtatataatacactataaagtagagaatggtgaagcaatatggaactatacatttgtaagattttcatgtattacatgaagtagaacaatgttaactgtaagtggactgtgaagagttaaggacgcatatgttaatttttagcacgattacttaaaaataatactaaaacagtatagctaaaaagtcaataggaaaattaaaatggaattctaaaaggtattcaagtttataaaaaaaggcaataaaaaagaaacacagaaacaacaaacagagaaaactaatataaaacaaataataaaatggtatactcacatycactcaagcaaagaattatgtcgcgtagtaattcattaaatactctaatcaaaggcagaggttgttagaatgaataaaaagaacaaaacccaaacatttgctatctataagtcatatgtttaaaatataaaaacagtgtggctgggaggcttcaagttgatccccatgatttgcgcctcttctagtcatgtccttgcataatcttttccccttgaatatcagcaggatccagggctttgttctaaccaatagattatggcaaaggtaaccagatggatgttattacatgtacatagttactgtacataagattgtaatgtccatcttgctaggaaactctctctccctccttggctttgaagacacatgctgccatgttatgagctgccatggaaggagtgccatcagcacaggatgtagggcagcctgtggctgatggccatcaagaaatggaggctcagagtcctacagcctgaaagaacccgaattctgccagcagtcacctaagcttggaatgagatccttccccagtggaacttctaatgagaacacggcccttgcaaacatcctgcttgcagacttgcagaggactcagtgaggtgtgtttggataacatagaaagtgtagatgatgcatgggttttattttaagaggctaaattttttatagtaatgttatacggtaaaagataactaatccagattttgatatctggaagtgggtgtattaatttgctagggctactgtaacaaaatatcttaaactagatggcttaaacaagagaaattcatcacctcacagttctgaaggctggaagtccaagaccaaggtactggcaatgctggtttcttctgagggttgtgagggagagctgatttcatgcctttcttctagcttctggtggtttactggaaatctttggcctttcttggcttatatatgtatcacctgatctctgccttcattttcttatggcattcttcctgtgtgcatgtctgcttctgtggataaatttcttctttttatgagagcatagtcatattgcattagagcccaccataaggaccttacattagcttaacctaatttgcaaagactctatttctaaataaagtcatacttacaggtattgggggttaggactccaatgtcttttcaggggaaatgattgaaacagtatcagtggttactaccgaaacaatacctaagagtgtggaagtgactttggaacaaggaagtgggaagactagaggattttgaggactgtgataaagaaagattaagttgccttgaactgacttagtagaaatctggaccttgaggaaactctcattgagggtgacagagaaggtgaggaacataggagtacttgtggaagagagacccatgttatgcagttgcagaaagcttagtgaaatttctcccttagttatgtttaaagaagaacacctaagtaataaaccttattatttaactaaggagatatccaagtaaagtgttgaaggtgctgcctgttccttttgctgtgtatagtaaaatgtgagaggaaagagataaattgaaagaagaaagattaagacaaaagcaaccaggacttgatgatttaaaaaatcttcagcctctccagatggcaaaagattctacaattaattaatgacttttgagcaaagtcagcaacaatcaggagaaatgctctttctttaactgtaaggagtacataagcttgaaattactggg

SEQ ID NO: 27 - DNA, 701 bp, Homo sapiens
gcttggcgctttgcagtcaggaatgaagcccacacccagccacaggcctgctcctgagacacagtgcagcctccatcaacggcacctggctcagacactcccgccagaatctctgccagccctgtccagaagccaggtgtggctgctcctgcactagccagaagggaacctgggcaggggcccctccaccgccaactcttggcccgacgaccggcgggctctgattggtagaggagctagaccaccagcgggttctgattggtgtacaaaacagattcccagagagagttctgatttgtggagctaatgtcacatgattaccctacagcaaaggatgctgggaagggaaatggcggctaccctagggaaaatttagatgtctaacctgggaaattcctcaaacctagaattcaggtttgccaggtggccgaaaaaaggacagatgtcccacacactgttgctcaggaaagacagggaagagtaaatgacacaatggctctrtagatgataaagagcaacacttttattattcttccagtggctccccaagtcccttaaacccgtgcccctcctcccgtcccccatgcaccctccaaaggagatgacaaaggcggcagtgaaggaggagaaggaggaagagaaacacctttattggaagagctgtgtggacaagagaacggggatgagagtgagagcatggg